(19) United States
(12) Patent Application Publication
(10) Pub. No.: US 2023/0301906 A1
(43) Pub. Date: Sep. 28, 2023

(54) BIOCOMPATIBLE, INJECTABLE AND IN SITU GELLING HYDROGELS AND PREPARATION AND APPLICATIONS OF BIOCOMPATIBLE, INJECTABLE AND IN SITU GELLING HYDROGELS BASED ON CELLULOSE NANOFIBRILS FOR TISSUE AND ORGAN REPAIR

(71) Applicant: OCEAN TUNICELL AS, Blomsterdalen (NO)

(72) Inventors: Christofer Troedsson, Paradis (NO); Eric Thompson, Paradis (NO); Hans Gude Gudesen, Luzern (CH)

(21) Appl. No.: 18/018,599

(22) PCT Filed: Jul. 30, 2021
(86) PCT No.: PCT/IB2021/000508

§ 371 Date: Jan. 30, 2023

Related U.S. Application Data
(60) Provisional application No. 63/059,342, filed on Jul. 31, 2020.

(51) Int. Cl. A61K 9/06; A61K 9/70; A61K 47/38; A61K 9/00

(52) U.S. Cl. CPC A61K 9/06; A61K 9/70; A61K 47/38; A61K 9/0019

(57) ABSTRACT

Hydrogels and preparation of biocompatible, in situ gelling and injectable hydrogels composed of cellulose nanofibrils and using them for tissue and organ repair and regeneration. The present invention demonstrates cellulose nanofibrils with tuned size (fibril length and distribution), high crystallinity, and high purity, variable concentration in water, and controlled charge for injectable hydrogels. The current application discovers that in situ gelling can be achieved by controlling the degree of substitution of cellulose nanofibrils through carboxymethylation. After injection, they can form three-dimensional gels and provide attachment sites and guidance for cells to facilitate tissue and organ repair. They can be also loaded with drugs, growth factors, or signaling molecules to be delivered to targeted locations in the body and released in a controlled manner. They can be mixed with animal or human cells, including stem cells, and provide for the transport of stem cells, cells, signaling molecules, and/or active components to injury sites.

### CROSS-REFERENCE TO RELATED APPLICATION(S)

The present application relies on the disclosures of and claims priority to and the benefit of the filing date of U.S. Provisional Patent Application No. 63/059,342, filed Jul. 31, 2020. The disclosures of that application are hereby incorporated by reference herein in their entireties.

### BACKGROUND OF THE INVENTION

### Field of the Invention

The present invention relates to biocompatible cellulose nanofibril-based water dispersions and their use as injectable hydrogels. These hydrogels are particularly suitable for injection in animals or humans, with or without the inclusion of drugs, growth factors, extracellular vesicles, or cells to repair or replace tissues or organs. Hydrogels, for example materials composed of a hydrophilic polymer network capable of holding large amounts of water, yet retaining structural integrity in preferred aspects, can be attractive biomaterials because they may be biocompatible and they can be tuned to deliver therapeutic agents to repair tissues and organs. In aspects, the present invention is capable of sufficiently resembling or including characteristics of natural extracellular matrices that provide sufficient structural support for cells to regenerate a tissue or organ. The characteristics of cellulose nanofibrils as explained herein with unique fibril structure, similar to dimensions of collagen in extracellular matrices, and presenting high purity and crystallinity, extreme water holding capacity, and shear thinning properties, make them preferable candidates for applications as injectable hydrogels for tissue and organ repair and regeneration. Not all biomaterials or hydrogels are suitable to be used for injection. For injectability, the biomaterial needs characteristics of a liquid with specific of sufficient viscosity for optimal or preferred flow. Most of the hydrogels based on synthetic polymers, such as polyethylene oxide, polypropylene oxide, or copolymers of those two, have relatively low viscosity and not an adequate or preferable effect of shear rate on viscosity. The same is generally true for hydrogels based on natural polymers, such as alginate or chitosan. The consequence of low viscosity is lack of control of injectability, thereby negatively affecting precision to reach a targeted tissue or organ site, as well as how effective the hydrogel will penetrate and shape inside the tissue or organ. Cellulose nanofibril-based water dispersions, such as described in present invention, have unique rheological properties (flow) characterized by solid like behavior at no shear and very strong shear thinning properties. This is ideal characteristic to satisfy good injectability. The rheological properties (flow), according to the present invention, can be varied, in aspects, by controlling the concentration of the nanocellulose dispersion and/or by, in aspects, controlling fibril length and/or fibril distribution during mechanical homogenization processes. The current invention shows and discovers that gelling after injection (e.g., in situ gelling) can be achieved by, in aspects, controlling the degree of substitution of cellulose nanofibrils through carboxymethylation. In addition, the surface interactions with drugs, growth factors, extracellular vesicles (“EVs”), or cells can be tuned by surface modifications of cellulose nanofibrils as described herein.

Such injectable hydrogels can be used for soft tissue repair and reconstruction, by way of example. Examples are, e.g., skin, fat tissue, or cartilage repair. The injectable hydrogels can, for example, also be used for wound healing and/or reconstruction of breasts, bone, or cartilage, as well as ligament repair. The injectable hydrogels can be loaded with growth factors such as Bone Morphogenetic Proteins (BMP) or TGF Beta and used for hard tissue repair such as bones in both medical and dental applications, in aspects. The injectable hydrogels can also be used to deliver drug and growth factors for spine repair or for cancer treatment, by way of example.

### Description of Related Art

Transplantable organ shortages are a serious, global problem. In the United States of America, around twenty people die every day waiting for transplants, and a new patient is added to the organ transplant list every 10 minutes (HRSA, USA). The situation of acute organ shortages causes high mortality rates of people that are on waiting lists or can lead people to attempt to obtain or obtain an organ through illegal and unethical pathways (WHO). The total cost of treating end-stage organ failure is estimated at $400 billion a year in the U.S. alone (1). Tissue engineering, for example in which biomaterials are combined with cells, offers an important alternative to help to resolve this global healthcare problem. Hydrogels, materials composed of a hydrophilic polymer network capable of holding large amounts of water, yet retaining structural integrity, are attractive biomaterials because most of them are biocompatible and they can be tuned to deliver therapeutic agents or cells to repair tissues and organs.

Biopolymers, including proteins such as collagen, and polysaccharides such as alginates, have been used as hydrogels for wound dressing, tissue engineering scaffolds, and drug delivery vehicles (2-5). Nanocellulose hydrogels have been shown to work as scaffolds for tissue engineering as they provide a combination of biocompatibility, fibril morphology, and water holding capacity (6-8). Basu et al. proposed the use of wood-derived nanofibrillated cellulose, NFC hydrogels for wound healing applications (9). In order to achieve self-standing hydrogels, they applied ion-induced crosslinking of the nanofibers. They have, however, only described carboxylated, and TEMPO oxidized NFC. In the current invention described herein, it has been found that carboxymethylated cellulose nanofibers have an increased ability to be divalent ion-induced crosslinked compared to TEMPO oxidized NFC; therefore, carboxymethylation, according to the current invention, is a possible method to modify the surface of the nanofibrils, by introducing carboxymethyl groups.

Rheological properties (viscosity and flow behavior) of cellulose nanofibril dispersions are affected by fibril length and fibril length distribution. Paakko et al. have described enzymatic hydrolysis combined with mechanical shearing and high-pressure homogenization for preparation of nanoscale cellulose fibrils from wood (10). However, it appears that they did not investigate the effect of homogenization cycles on fibril lengths and rheological properties.

Most injectable hydrogels reported in the literature have relatively low stiffness/robustness which is a limitation in long-term applications for tissue and organ repair. Yang et al. have used cellulose nanocrystals to reinforce carboxymethylcellulose hydrogels (11). De France et al. described use of cellulose nanocrystals to reinforce Poly(oligo Ethylene Glycol Methacrylate) injectable hydrogels (12).

Recent reviews describe potential uses of cellulose nanofibril hydrogels in biomedical applications with emerging use as bioinks for 3D bioprinting and cell culture support materials (13-15). In U.S. Pat. No. 10,675,379B2, issued on Jun. 9, 2020, cellulose nanofibrillar hydrogels are proposed to be used as bioinks for 3D bioprinting, cell culturing, tissue engineering and regenerative medicine applications (16). The reference describes different mechanical, enzymatic and chemical steps to yield dispersions of cellulose nanofibrils in liquid media, wherein the cellulose nanofibrils have a length of about 1-100 microns and a width of about 10 nanometers to 20 microns; with desired morphological and rheological properties to be used as bioinks in 3D Bioprinter applications. 3D Bioprinted samples must be crosslinked prior to use and then the constructs need to be surgically implanted. This requires the presence of bioprinters and additional skilled personnel in the operating theatre, adding to the complexity and costs of the interventions. The current invention improves upon the more invasive surgical procedure of implantation and describes direct injection of the hydrogel without the necessity to use bioinks and 3D bioprinting, in aspects.

In another reference, a 3D discontinuous entity for culturing of cells is described, comprising an aqueous medium and hydrogel bodies including cellulose nanofibrils and/or derivatives thereof, suspended in the aqueous medium (17). In another patent, U.S. Pat. No. 10,612,003, plant-derived cell culture materials for cell culture or cell delivery, comprising sterile, mechanically disintegrated cellulose nanofibers and/or derivatives thereof, in the form of a 3D hydrogel matrix having a nanofiber concentration ranging from about 0.01 to 1.7 wt %, wherein the cellulose nanofibers and/or derivatives thereof are structurally type I cellulose, and a plurality of cells homogeneously distributed within the three-dimensional matrix, is described (18). Both patents only describe plant nanocellulose hydrogels for in vitro cell culturing.

In summary, there is an emerging need for biocompatible, injectable, in situ gelling hydrogels with robust mechanical properties for tissue and organ repair. Tunicate cellulose nanofibril hydrogels prepared as described in the present invention, will be preferred candidates for injectable hydrogels for tissue and organ repair.

Thus, the current invention improves upon the art and describes the preparation of biocompatible, injectable, in situ gelling hydrogel formulations based on, in aspects, tunicate cellulose nanofibrils for animal and human tissue and organ repair and regeneration. Injectable hydrogels are attractive for tissue and organ repair because in contrast to surgically implanted materials they exhibit minimally invasive delivery procedures, which reduces healing time, lowers costs for hospitals, generates less pain for patients, reduces scarring, and decreases risks of post-operative infections.

### SUMMARY OF THE INVENTION

In the current invention, preparation of biocompatible, injectable, in situ gelling hydrogels composed of cellulose nanofibrils, preferably, in embodiments, derived from tunicates, is taught; including for applications in tissue and/or organ repair. After injection, in aspects, hydrogels composed of cellulose nanofibrils with tuned size (fibril length and distribution), crystallinity, concentration, chemical composition of the surface, and electrical charge, form three-dimensional (“3D”) matrices providing attachment sites and guidance for cells, to facilitate tissue and/or organ repair. They can also be pre-loaded with drugs, growth factors, or signaling molecules, enabling delivery to targeted locations in the body, with rates of release tuned by the matrix properties to optimize efficacy, in aspects. They can be mixed with animal or human cells, including stem cells, to provide the transport of stem cells to injury sites. Instead of using cells or stem cells, extracellular vesicles (EVs) or autologous tissue aspirates can also be used to stimulate repairs undertaken by endogenous cells. Here, biocompatible cellulose nanofibril dispersions can be used as a main component of injectable hydrogels. The current invention describes tuning the length and length distribution of cellulose nanofibrils by mechanical treatment in homogenizers, and adjusting viscosity by selecting a range of fibril concentrations, to provide desired viscosity and shear thinning properties for controlled injectability. The surfaces of cellulose nanofibrils may be modified by carboxymethylation to control surface electrical charge and enable in situ gelling properties. An advantage of using cellulose nanofibrils derived from, for example, tunicates as a component of injectable hydrogels is, by way of example, a combination of beneficial rheological properties such as shear thinning and fast recovery (flow through various tissues after injection), in situ gelling by crosslinking with endogenous calcium or other divalent cations in physiological environments, and/or biocompatibility coupled to 3D architectures favorable for cell attachment and growth. These features show injectable hydrogels based on cellulose nanofibril dispersions to be functional injectable hydrogels for tissue and/or organ repair.

In embodiments, the current invention is capable of imparting preferable properties in a hydrogel, including, but not limited to, one or more of the following:
1. Biocompatibility;
2. Water holding capacity and retention;
3. Rheological properties (suitable viscosity, shear thinning and cavity filling capacity);
4. Controlled in situ gelling;
5. Controlled and targeted interactions with other materials such as drugs, growth factors, conductive components, and cells; and
6. Controlled in vivo function.

### BRIEF DESCRIPTION OF THE DRAWINGS

The accompanying figures illustrate certain aspects of some of the embodiments of the present invention, and should not be used to limit or define the invention. Together with the written description, the drawings serve to explain certain principles of the invention.

FIG. 1a shows AFM images of fibril homogenized with 9 cycles and FIG. 1b and FIG. 1c show normal distributions of fibril lengths at different degrees of homogenization cycles.

FIG. 2 shows the effect of shear rate on viscosity for hydrogels homogenized with various numbers of cycles.

FIG. 3 shows the effect of mechanical treatment on injectability in 2% and 10% gelatin as models of differing tissue densities.

FIG. 4 shows the effect of cellulose nanofibril concentration on rheological properties.

FIG. 5 shows the effect of cellulose nanofibril concentration on injectability in soft tissue models composed of 2% and 10% gelatin.

FIG. 6 shows gelling experiments for alginate and various tunicate cellulose nanofibril hydrogels.

FIG. 7 shows the experimental set-up to study the gelling process.

FIG. 8 shows the effect of addition of a crosslinking agent, in this example 100 mM calcium chloride, on gelling of selected hydrogels. The crosslinking agent was added after 60 seconds in this example.

FIG. 9 shows the release profile of hemoglobin from crosslinked TUNICELL-alginate hydrogels with various compositions.

### DETAILED DESCRIPTION OF VARIOUS EMBODIMENTS OF THE INVENTION

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Embodiments comprising various features may also consist of, or consist essentially of, those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention. All references cited in this specification are hereby incorporated by reference in their entireties.

The present invention demonstrates cellulose nanofibrils with, in aspects, tuned size (fibril length and distribution), high crystallinity, high purity, variable concentration in water, and controlled charge for injectable hydrogels. The current application shows that in situ gelling can be achieved by controlling the degree of substitution of cellulose nanofibrils through carboxymethylation. After injection, they can form three-dimensional gels and provide attachment sites and guidance for cells to facilitate tissue and organ repair and/or generation and/or regeneration. They can also include one or more pharmaceutical, growth factor, or signaling molecule, for example, to be delivered to targeted locations in the body and released in a controlled manner. They can be mixed with animal or human cells including stem cells and provide for the transport of stem cells or cell signals to injury sites, by way of example.

The rheological properties (flow) can be varied, in aspects, by controlling the concentration of the nanocellulose dispersion and/or by, in aspects, controlling fibril length and/or fibril distribution during mechanical homogenization processes. In embodiments, fibril length and/or distribution may be tuned by altering the number of cycles in the mechanical homogenization process. This will change the rheological properties of the hydrogel and facilitate injectability, in situ gelling, and/or cell viability. Additional cycles in the mechanical homogenization will, in aspects, reduce fiber length, which will lower viscosity and result in improved injectability of the hydrogel.

In embodiments, a difference in crystallinity and/or purity can be achieved by using different sources of cellulose. Tunicate nanocellulose has a preferable crystallinity and purity. In aspects, a higher degree of crystallinity can facilitate the mechanical property or properties of the hydrogel, such as retaining specific and desirable shapes. In aspects, a higher degree of purity can enhance biocompatibility and tissue integration in animals and/or humans for tissue and organ repair application(s).

In embodiments, the current invention is able to allow for variable concentration of the cellulose nanofibrils in water or other liquid or solution. Higher concentration of cellulose nanofibrils will, in aspects, yield higher viscosity of the hydrogel as well as changing the porosity for diffusion of cell signals or active compounds preloaded into the hydrogel. The concentration of the cellulose nanofibril dispersion can therefore be used to tune preferable flow properties of the hydrogel for enhanced cell viability, specific diffusion rates and/or tissue requirements.

In embodiments, an electrical charge of the cellulose fibrils can be controlled by varying the degree of substitution. This substitution is a surface modification of the cellulose fibrils wherein, by way of example only, carboxyl or carboxymethyl groups are added to the fibrils. Using different numbers or amounts of carboxyl or carboxymethyl groups will alter the surface properties, such as charge, and the cellulose fibrils' capability of crosslinking to, for example, molecules and fibers in the hydrogel, which can thereby impact its crosslinking kinetics. Adding carboxyl or carboxymethyl groups can also allow for further modification through grafting functional molecules to the cellulose fibers in the hydrogel, which can facilitate functionality in specific applications.

The current application shows that in situ gelling can be achieved by, in aspects, controlling the degree of substitution of cellulose nanofibrils through carboxymethylation. And, in aspects, carboxymethylated cellulose nanofibrils can exhibit an increased ability to be divalent ion-induced crosslinked as compared to TEMPO oxidized cellulose nanofibrils, for example; therefore, carboxymethylation, according to the current invention, is a possible method or mechanism to modify the surface of the nanofibrils, by introducing carboxymethyl groups.

In embodiments, hydrogels according to the present invention can also be pre-loaded with drugs, growth factors, or signaling molecules, enabling delivery to targeted locations in the body. In aspects, rates of release are tuned by the matrix properties to optimize efficacy and/or safety. In embodiments, the surface interactions with drugs, growth factors, extracellular vesicles, cells and autologous tissue aspirate can be tuned by surface modifications of cellulose nanofibrils as described herein. By varying the concentration of nanofibrils in the hydrogel, variable porosities can be achieved, which can alter diffusion of active molecules from the hydrogel into adjacent tissue, organs, and/or body parts after injection, in aspects. Chemically modifying the nanofibers by, such as, but not limited to, adding carboxyl and/or carboxymethyl groups, can also allow for grafting functional components into the hydrogel. By modifying the nanocellulose via, e.g., periodate oxidation, oxidized cellulose nanofibrils can be bioconjugated to other biopolymers, such as, but not limited to, fibronectin, laminin, and collagen, for enhanced cell interaction.

In embodiments, a hydrogel is injected into a wound, tissue, body part, or organ and crosslinked in situ, by adding one or more crosslinking molecules, such as a divalent cation, simultaneously during injection, nearly simultaneously during injection, substantially simultaneously during injection, about simultaneously during injecting, or temporally separated, to the hydrogel. The hydrogel may also be crosslinked by the wound's, tissue's, body part's, or organ's physiological conditions or properties, such as endogenous calcium concentration. In embodiments, the in situ gelling allows for injection, such as a smooth, comfortable, effective, medically efficacious, and/or medically safe injection, of the hydrogel with or without active components, cells, or ingredients, and then allows for molding or shaping of the injected hydrogel to a desired form(s) inside the wound, tissue, body part, or organ. In embodiments, the hydrogel comprises a mechanical strength, such as a high mechanical strength for example, to facilitate the in situ molding or shaping so that the hydrogel can retain the desired shape indefinitely, permanently, temporarily, or for a period of time. Cellulose nanofibers according to the current invention can have the mechanical properties to allow in situ molding and shaping. The in situ crosslinking can allow the injected hydrogel's shape to be retained.

In aspects, the in situ gelling comprises hydrogel formation or gelation at the site of injection and/or once the hydrogel has been injected. In aspects, the gelling can be self-gelling or created upon addition of a crosslinking molecule(s) such as, but not limited to, divalent cations such as calcium. For example, in aspects, the gelling would occur when or after the hydrogel is injected into, for example, a human or animal tissue, body part, organ, or wound. In aspects, this allows for working with hydrogels without having to perform more invasive surgical procedures. In aspects, the gel becomes hardened, hard, rigid, semi-rigid, more viscous, elastic, semi-elastic, soft, or semi-soft when or after the hydrogel is injected. In aspects, in situ gelling comprises lower viscosity of the hydrogel prior to injection and, once injected, becoming more viscous or a gel at the injection site or internally, such as in vivo, by way of example only. In addition to other applications, this can allow the injected hydrogel to form a scaffolding upon injection of the hydrogel. In aspects, in situ gelling comprises the hydrogel being less viscous for purposes of injecting and then more viscous when or after the hydrogel is injected. In aspects, the hydrogel can gel slowly, semi-slowly, rapidly, semi-rapidly, spontaneously, or nearly spontaneously upon injecting, when injecting, or after injecting. In aspects, hydrogel formation occurs in situ, with or without the aid, inclusion, or addition of crosslinking agents. In aspects, crosslinking agents can be included in or added to the hydrogel either before, during, or after injection. In aspects, the gel is macroscopic or microscopic. In aspects, the gelling is reversible or non-reversible. In aspects, the gel consists of cellulose nanofibril dispersion alone, or with one or more polymers, biopolymers, active components, cells, stem cells, cell signals, or EVs. In aspects, the cellulose nanofibrils are chemically modified, such as by carboxymethylation, TEMPO oxidation, periodate oxidation, or enzymatic treatment. In aspects, the cellulose fibers are bioconjugated with other biopolymers such as, but not limited to, collagen, laminin, and/or fibronectin.

To facilitate a better understanding of the present invention, the following examples of certain aspects of some embodiments are given. In no way should the following examples be read to limit the scope of the invention.

### Example 1

Effect of Mechanical Pre-Treatment on Injectability

Dispersions of enzymatically pretreated tunicate cellulose nanofibril s, TUNICELL ETC, were homogenized in a high-pressure fluidizer (Microfluidizer M-110EH, Microfluidics Corp. USA) using various numbers of cycles (passes through the homogenizer). ETC dispersions were then evaluated with regards to fibril length distributions using Atomic Force Microscopy (AFM) (8). A freshly cleaved mica sheet was treated with a solution of poly-L-lysine (0.01%) for 5 minutes and then air dried. A drop of diluted ETC dispersion (0.02% dry content) was then deposited on the mica and incubated for 5 minutes, followed by rinsing with DI water. The dried samples were examined with an AFM NanoScope III scanning probe microscope with a type G scanner equipped with Nanoscope software (v.4.43; Digital Instruments, Santa Barbara, CA, USA). Measurements were performed in tapping mode with a standard silicon tip (height: 15 μm; curvature radius: 8 nm) to determine the length and width of the nanocellulose fibrils, which were then calculated using ImageJ (National Institutes of Health, Bethesda, MD, USA) on an average of 5-10 fibrils. The rheological properties of the ETC were assessed with a TA Discovery HR2 rheometer (TA Instruments, New Castle, DE, USA) with a peltier aluminum plate (20 mm in diameter; gap=300 μm). To determine the linear viscoelastic region (LVR), oscillation amplitudes were set to a range of 0.1 Pa to 1000 Pa at a frequency of 1 Hz. From the LVR, a force of 10 Pa was chosen for the oscillation-frequency measurements at a range of 10⁻³ Hz to 10³ Hz. Shear viscosity was evaluated by increasing the shear rate from 0.1 s⁻¹ to 1000 s⁻¹ at 25° C.

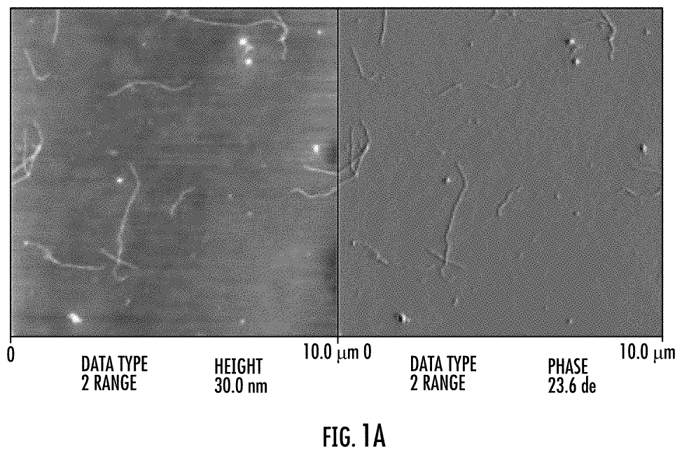

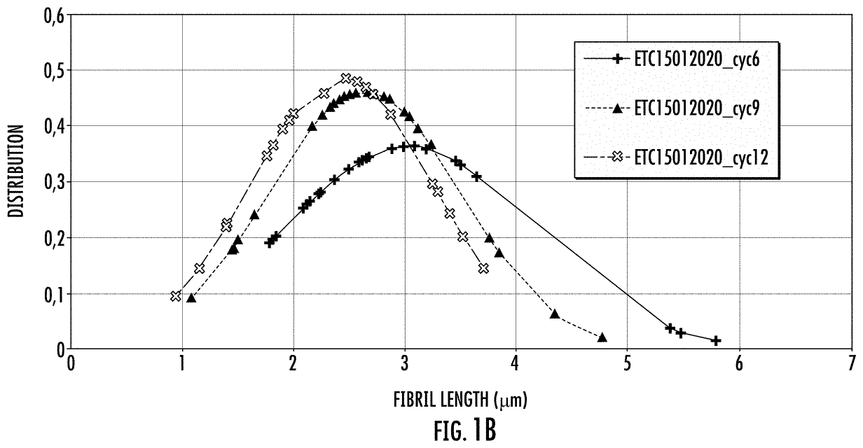

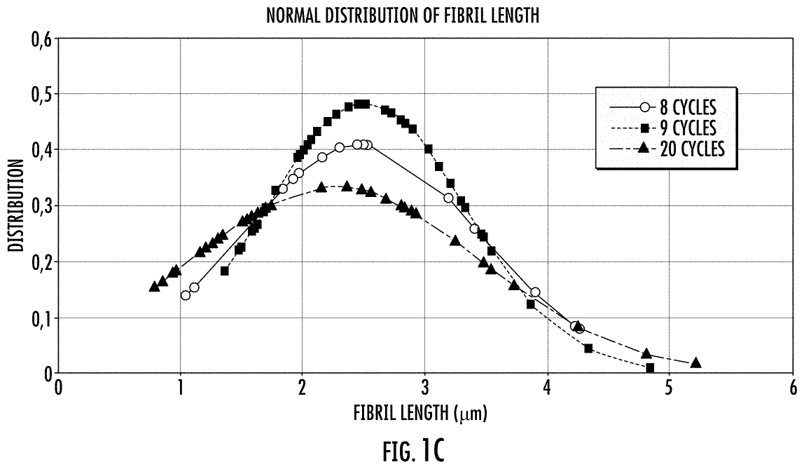

Table 1 summarizes results from AFM fibril length determinations. The average fibril length for the mechanical treatment with 6 cycles was above 3 μm. The average fibril length decreased to 2.64 μm when run through 9 cycles and decreased further to 2.43 after 12 cycles of homogenization. The fibril dimensions analyzed by AFM after 9 cycles are shown in FIG. 1a and fibril size distributions are shown in FIG. 1b. It is important to note that the mechanical treatment by homogenization decreased the average fibril length. Increasing mechanical treatment to 20 cycles further reduces average fibril length to 2.27 micrometers, but the fibril size distribution became again broader, probably by formation of fine material (see FIG. 1c).

TABLE 1
Characteristics of different preparations of enzymatically
treated tunicate cellulose nanofibrils.

| | | Average Fibril | |
|---|---|---|---|
| | Concentration | length of ETC | Viscosity of |
| Sample | of ETC | (micrometer) | ETC at 1 1/s |
| ETC 6 cycles | 2.55% | 3.03 | 144 Pa · s |
| ETC 9 cycles | 2.59% | 2.64 | 147 Pa · s |
| ETC 12 cycles | 2.61% | 2.43 | 93 Pa · s |
| ETC 20 cycles | 2.41% | 2.27 | 61 Pa · s |

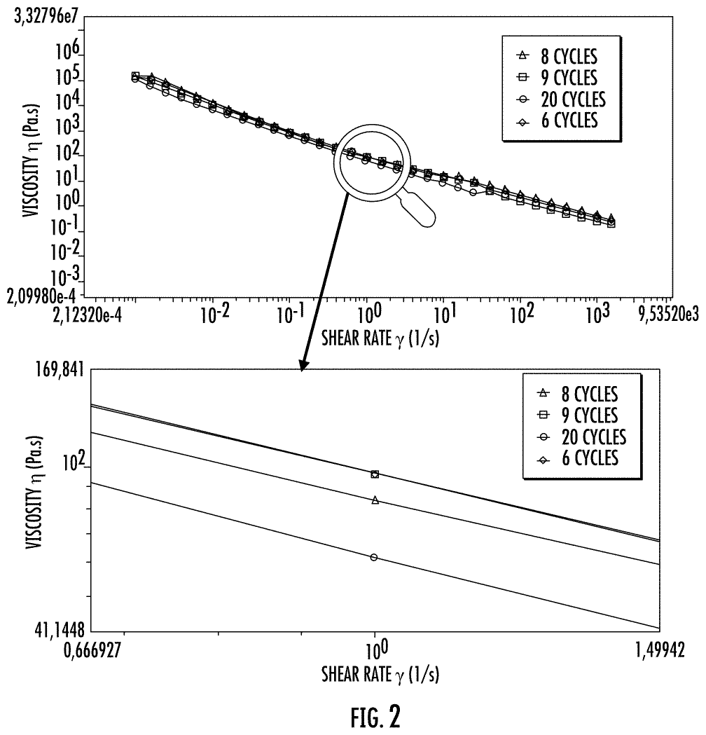

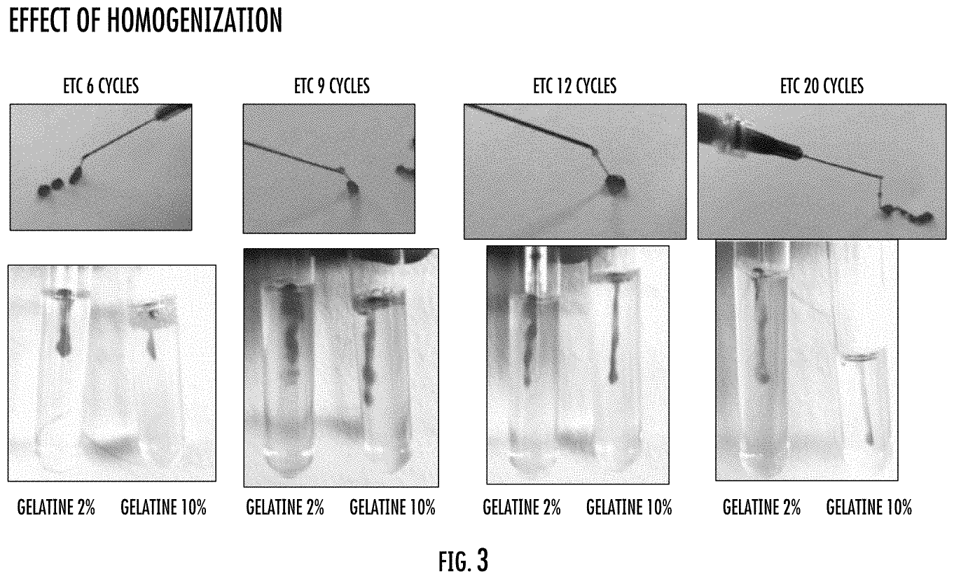

FIG. 2 shows the effect of homogenization with various numbers of cycles on rheological properties of cellulose nanofibril hydrogels. Hydrogels have shear thinning properties, which means generally that increased shear rate results in lower viscosity. The lower figure panel shows the magnified area at shear rate of 1×1/s. At this magnification it is easier to see that the homogenization with 20 cycles results in lower viscosity at all shear rates. This is also seen in Table 1. The injectability of different hydrogels was compared by coloring with an oil-based red dye and observing the shape of the hydrogels emerging from the needle. In the upper row of panels in FIG. 3 it can be seen that hydrogels homogenized through 6 cycles formed droplets when dispensed. Increased homogenization resulted in relatively better flow when dispensed through the needle. At 20 cycles, in this example, the dispersion flowed relatively more smoothly through the needle. Also studied and tested was the injectability of these hydrogels by simulation of soft tissue. Gelatin gels were cast with two different concentrations; 2% and 10% to simulate tissues with different densities. The hydrogels were then dispensed with a needle inserted at a top of a vial with a gelatin matrix (see lower panels in FIG. 3). The evaluation was made by qualitative comparison of the length, width, and shape of the injected hydrogel after it had entered into the gelatin matrix. A trend was seen showing improved injectability with increased homogenization. The hydrogel homogenized with 20 cycles exhibited straight long tracks with the narrowest width compared with hydrogels homogenized at lower cycles numbers.

### Example 2

Effect of Concentration on Injectability

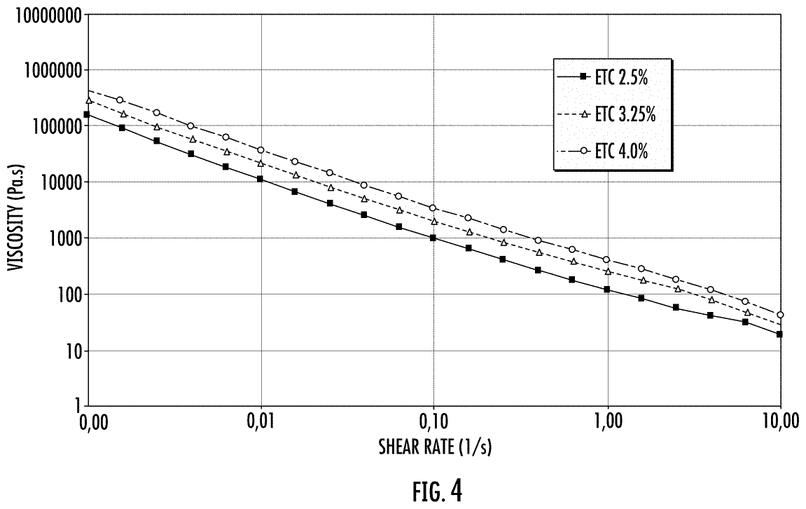

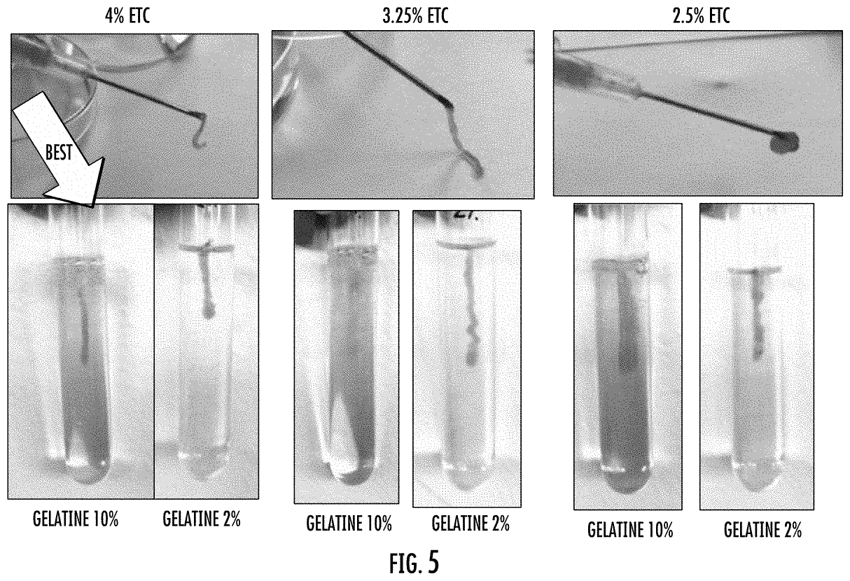

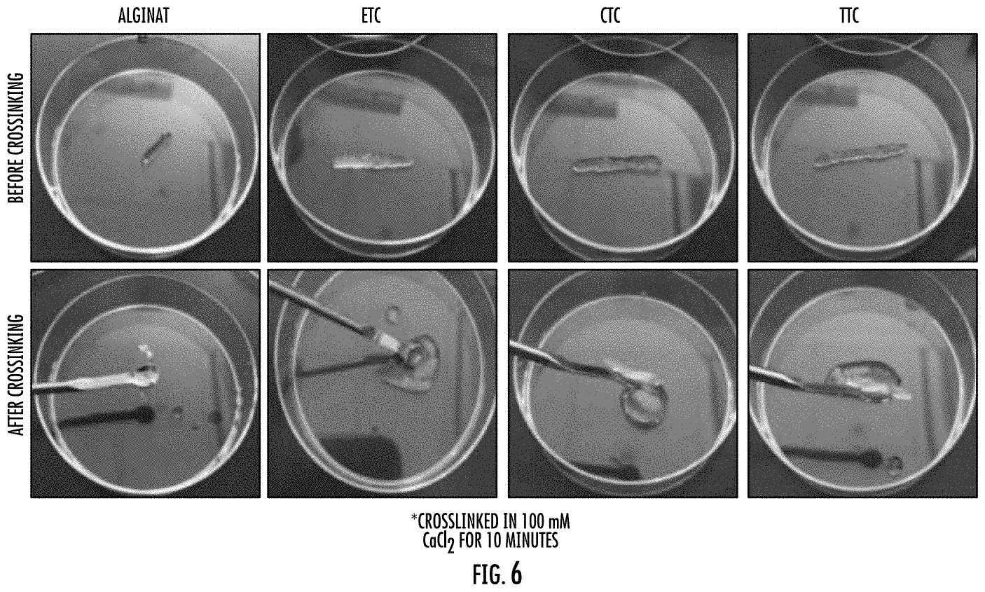

Dispersions of enzymatically pretreated tunicate cellulose nanofibrils, TUNICELL ETC, produced with homogenization at 9 cycles, was post-treated to increase the concentration. Post-treatment included vacuum filtration. The concentration was increased from 2.5% to 3.25% and to 4%. The effect of concentration on the viscosity-shear rate relationship was investigated using a rheometer under conditions as described in Example 1. FIG. 4 shows that all three hydrogels exhibited shear thinning with viscosity being higher at higher nanocellulose concentrations over the entire range of shear rates range that were investigated. The injectability was compared by visual inspection of hydrogels dispensed from a 20-gauge syringe needle and by comparing the shape of dispensed hydrogels in 2% and 10% gelatin soft-tissue models (FIG. 5). By way of an example, a conclusion was reached that a hydrogel with higher or highest concentration had preferred injectability.

### Example 3

In Situ Gelling

In situ gelling ability of cellulose nanofibril dispersions was investigated by crosslinking with calcium chloride dispensed hydrogels with different surface charges and comparing it to alginate. Three different cellulose nanofibril hydrogels were selected for the study: Enzymatic tunicate cellulose, ETC; Carboxymethylated tunicate cellulose, CTC; and TEMPO oxidized tunicate cellulose, TTC. The surface charge was determined using zeta potential measurements (*-potential, i.e., the average charge of the fibrils) using DLS (Nano ZS-ZEN3600; Malvern Instruments, Malvern, UK).

CTC samples were subjected to conductometric titration and the charge density was 367 μmol/g, which corresponds to a degree of substitution (DS) of 0.062. The charge density of the TEMPO oxidized sample, TTC was 664 μmol/g. Alginate (Pronova SLG100 from Nova Matrix, Norway) was used for comparison. Table 2 summarizes results of zeta potential measurements.

TABLE 2
Zeta potentials of different tunicate
cellulose nanofibril preparations.

| Material | z-Potential (mV) |
|---|---|
| ETC | −16.9 |
| CTC | −57.2 |
| TTC | −62.5 |

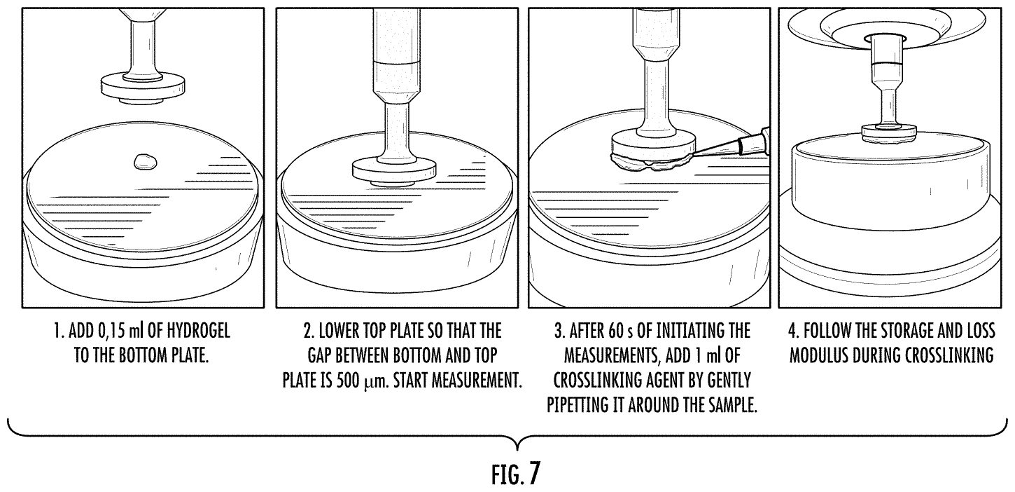

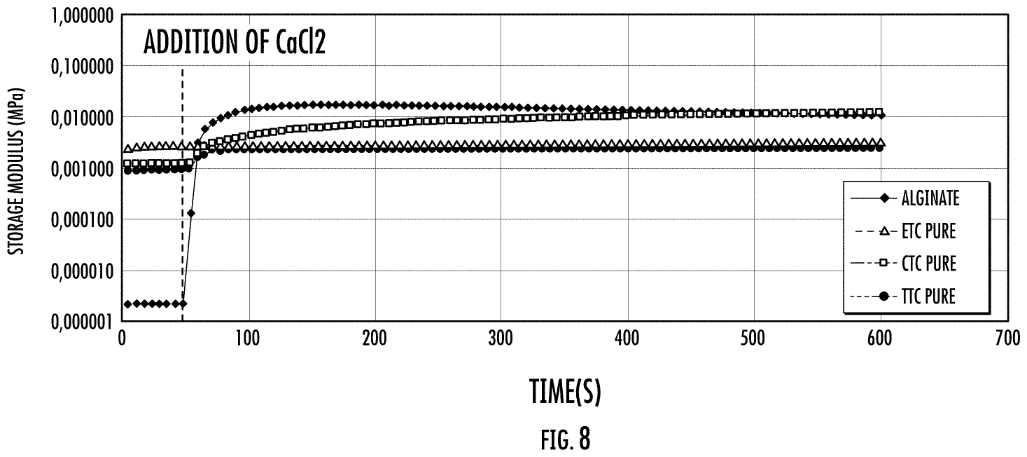

The crosslinking solution was 100 mM calcium chloride in DI water. Gelling was screened by dropping the calcium chloride solution onto matrices dispensed through 20-gauge needles. Alginate sample was evaluated as a 3% solution in DI water. Gelling was then studied with oscillation-time measurements at 1.5% strain and a frequency of 1 Hz for 10 minutes using a Discovery HR-2 rheometer (TA Instruments, Crawley, UK). All measurements were conducted at 25° C., with a plate-plate geometry of 20 mm (gap: 500 μm). At 60 seconds after initiating the measurement, 1 ml of 0.1 M CaCl₂ was dispensed around the sample while gathering data on the storage and loss moduli. FIG. 7 shows the experimental set-up to study gelling of hydrogels. FIG. 8 shows the effect of adding a crosslinking agent, 100 mM calcium chloride solution, on the gelling of selected hydrogels. Crosslinking agent was added after 60 seconds. The storage modulus in shear mode, which describes the stiffness of the hydrogel, is displayed as a function of time. At time 0, one can observe a difference between the alginate solution, which is not a hydrogel before crosslinking, and different TUNICELL hydrogels. ETC material, which has not been modified, has a higher or highest storage modulus followed by carboxymethylated TUNICELL (CTC) and then TEMPO oxidized TUNICELL (TTC). After addition of 100 mM calcium chloride solution, the storage modulus of alginate increased due to rapid crosslinking and in cases immediately increased. The crosslinked hydrogel had higher or the highest stiffness of the analyzed hydrogels, in cases directly after addition of the crosslinking agent. Unmodified TUNICELL ETC in cases could be affected but in cases were not substantially affected by addition of the crosslinking agent. TTC hydrogels showed a moderate increase in storage modulus and achieved equilibrium, in cases quickly or rapidly achieving equilibrium. CTC hydrogel showed a relatively slower increase in the rate of storage modulus, but after 500 seconds, exhibited a storage modulus preferable to alginate. This shows that carboxymethylation is a suitable method of modification of cellulose nanofibril dispersions to provide crosslinking ability and in situ gelling properties. Table 3 summarizes the storage modulus of the analyzed materials 540 seconds after addition of 100 mM calcium chloride solution. CTC had the highest storage modulus among analyzed materials.

TABLE 3
Storage Moduli of alginate and different
tunicate nanocellulose preparations

| | Storage Modulus |
|---|---|
| | (Pa) (at 10 min) |
| Alginate | 10948 |
| ETC pure | 3120 |
| CTC pure | 12147.5 |
| TTC pure | 2360 |

### Example 4

Biocompatibility

TUNICELL hydrogels were processed and further refined using a high-pressure fluidizer in a validated cleanroom facility. The processing protocols resulted in highly crystalline, high aspect ratio, >99% pure cellulose, free of contaminating hemicellulose and lignin. Table 4 summarizes the carbohydrate composition of TUNICELL. Released carbohydrates after complete acid hydrolysis of TUNICELL were examined by high performance anion exchange chromatography with a pulsed amperometric detection (HPAEC-PAD) on a ICS3000 system (Dionex, Sunnyvale, CA, United States) using a Carbopac PA1 column (Dionex, Sunnyvale, CA, United States). Medical grade ultrapure TUNICELL produced under clean room standards, was electron beam sterilized and had bioburden levels of <10 CFU/ml and endotoxin levels ≤0.5 EU/ml, adhering to FDA regulations for implantable devices. Endotoxin levels of TUNICELL were tested using Lonza's PyroGene™ Recombinant Factor C Assay. The bioburden test was conducted according to European Pharmacopoeia, Chapter 2.6.12.

TABLE 4
Carbohydrate composition of TUNICELL (tunicate cellulose)

| Carbohydrate | Relative composition (%) |
|---|---|
| Arabinose | 0.3 ± 0.02 |
| Galactose | <0.1 |
| Glucose | 99.2 ± 0.3 |
| Xylose | <0.1 |
| Mannose | 0.4 ± 0.1 |

### Example 5

Hemoglobin Delivery

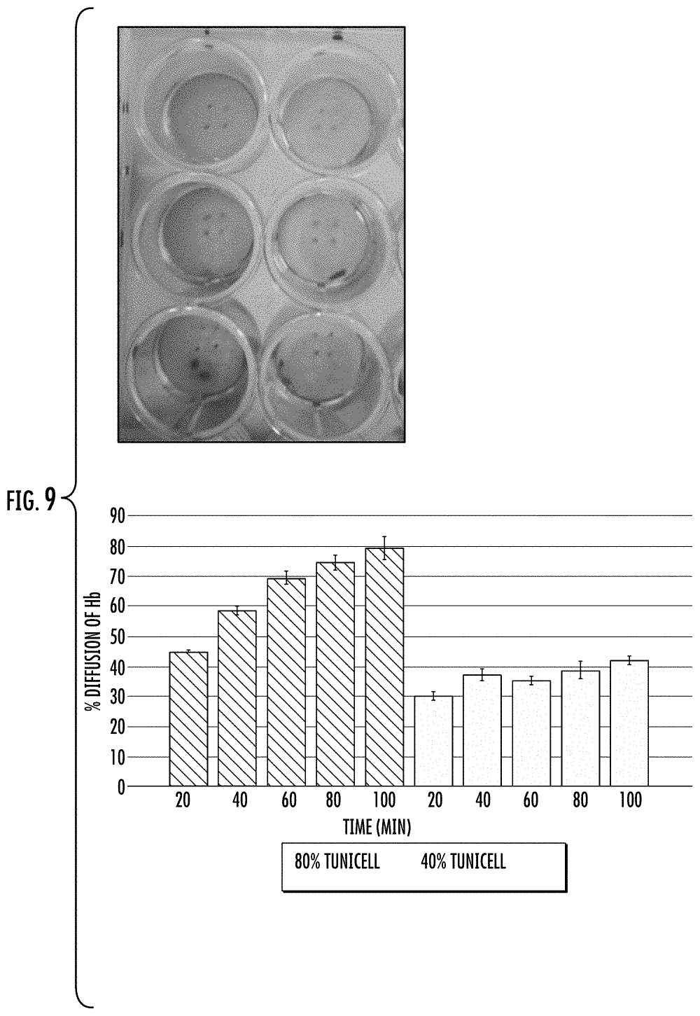

TUNICELL hydrogels were also evaluated for drug and growth factor delivery. As an example, Hemoglobin delivery was evaluated in order to enhance tissue oxygenation and thus accelerate the wound healing process. Two different hydrogels with TUNICELL-alginate mixtures at 80:20 and 40:60 were prepared. The hydrogels, crosslinked using 100 mM calcium chloride solution, were loaded with hemoglobin by soaking crosslinked hydrogels in hemoglobin solution. The hemoglobin release rate was evaluated by determination of hemoglobin concentration using UV spectroscopy when placing crosslinked hydrogel in HBSS solution. Changing the composition of the hydrogel by varying ratios between TUNICELL and alginate allowed differential diffusion rates of hemoglobin adjusted and adjustable to wound and wound healing requirements (FIG. 9). A faster release of hemoglobin was seen with hydrogel with higher concentration of TUNICELL. Accordingly, it was discovered that this is an applicable method to dispense and deliver hemoglobin to wounded tissue in a controlled manner.

### Example 6

Enhancing Cell Interactions

TUNICELL hydrogel, ETC was modified by periodate oxidation followed by bioconjugation of selected extracellular proteins in order to control cell adhesion. In a glass bottle covered with aluminum foil containing 15 g of ETC (2.4% concentration), a solution of 0.73 g of sodium periodate (1.5 mole of periodate/anhydrous glucose unit) in 5 mL of DI water was added. The reaction was stirred for 24 hours at room temperature and the hydrogel was then centrifuged and rinsed with DI water. The oxidized constructs were then bioconjugated with fibronectin, collagen I and laminin by adding between 1 and 15 ml of 100 μg protein per ml solutions to oxidized hydrogel, followed by incubation at 37° C. for 24 hours. The bioconjugated hydrogels were then rinsed briefly in deionized water and centrifuged to a desired concentration. The bioconjugated hydrogels were used with and without cells for injection into soft tissue to repair defects. The bioconjugated hydrogels showed enhanced cell adhesion which contributed to tissue repair and improved tissue repair. The bioconjugated hydrogels were mixed with human chondrocytes and injected into joints for cartilage repair. After 28 days, human cartilage was developed within regions where hydrogels were implanted.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention. Algae as used herein includes but is not limited to macroalgae and microalgae, as well as all forms of algae.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

### REFERENCES

As noted above, the following references are incorporated by reference herein in their entireties.
1. WHO, organ failure. And E. Caló, V. V. Khutoryanskiy, Biomedical applications of hydrogels: A review of patents and commercial products, European Polymer Journal 65 (2015) 252-267.
2. J. A. Rowley, G. Madlambayan, D. J. Mooney, Alginate hydrogels as synthetic extracellular matrix materials, Biomaterials 20 (1) (1999) 45-53.
3. K. Y. Lee, D. J. Mooney, Alginate: properties and biomedical applications, Prog. Polym. Sci. 37 (1) (2012) 106-126.
4. D. J. Overstreet, D. Dutta, S. E. Stabenfeldt, B. L. Vernon, Injectable hydrogels, J. Polym. Sci. B: Polym. Phys. 50 (13) (2012) 881-903.
5. S. Van Vlierberghe, P. Dubruel, E. Schacht, Biopolymer-based hydrogels as scaffolds for tissue engineering applications: a review, Biomacromolecules 12 (5) (2011) 1387-1408.
6. G. Helenius, H. Bäckdahl, A. Bodin, U. Nannmark, P. Gatenholm, B. Risberg, In vivo biocompatibility of bacterial cellulose, J. Biomed. Mater. Res. A76 (2006) 431-438.
7. Backdahl, H.; Helenius, G.; Bodin, A.; Nannmark, U.; Johansson, B. R.; Risberg, B.; Gatenholm, P. Mechanical properties of bacterial cellulose and interactions with smooth muscle cells. Biomaterials 2006, 27 (9), 2141-9.
8. Apelgren, P., Karabulut, E., Amoroso, M., Mantas, A., Martinez Ávila, H., Kolby, L., Kondo, K., Toriz, G., and Gatenholm, P., 2019. In Vivo Human Cartilage Formation in Three Dimensional Bioprinted Constructs with a Novel Bacterial Nanocellulose Bioink, ACS Biomater. Sci. Eng. 5, 2482-2490.
9. Basu A, Lindh J, Ådander E, Strømme M, Ferraz N. On the use of ion-crosslinked nanocellulose hydrogels for wound healing solutions: physicochemical properties and application-oriented biocompatibility studies. Carbohydr Polym 2017; 174: 299-308.
10. Paakko M, Ankefors M, Kosonen H, et al. Enzymatic hydrolysis combined with mechanical shearing and high pressure homogenization for nanoscale cellulose fibrils and strong gels. Biomacromolecules 2007; 8(6): 1934-1941.
11. Yang, X.; Bakaic, E.; Hoare, T.; Cranston, E. D. Injectable Polysaccharide Hydrogels Reinforced with Cellulose Nanocrystals: Morphology, Rheology, Degradation, and Cytotoxicity. Biomacromolecules 2013, 14 (12), 4447-4455.
12. De France, K. J.; Chan, K. J. W. W.; Cranston, E. D.; Hoare, T. R. Enhanced Mechanical Properties in Cellulose Nanocrystal-Poly(oligo Ethylene Glycol Methacrylate) Injectable Nanocomposite Hydrogels through Control of Physical and Chemical Cross-Linking. Biomacromolecules 2016, acs.biomac.5b01598.
13. Du H, Liu W, Zhang M, Si C, Zhang X, Li B., Cellulose nanocrystals and cellulose nanofibrils based hydrogels for biomedical applications. Carbohydr Polym. 2019 Apr. 1; 209:130-144. doi: 10.1016/j.carbpol.2019.01.020.
14. del Valle J L, Díaz A, Puiggali J. Hydrogels for biomedical applications: cellulose, chitosan, and protein/peptide derivatives. Gels 2017; 3:1-28.
15. Curvello R, Raghuwanshi V S, Gamier G, Engineering nanocellulose hydrogels for biomedical applications. Advances in Colloid and Interface Science 267 (2019) 47-61.
16. U.S. Pat. No. 10,675,379B2 Cellulose nanofibrillar bioink for 3D bioprinting for cell culturing, tissue engineering and regenerative medicine applications.
17. Lou Y. R. et al., The use of Nanofibrillar cellulose hydrogel as a flexible three-dimensional model to culture human pluripotent stem cells, Stem cell and development 2014; Vol. 23:4, pp. 380-392.
18. U.S. Pat. No. 10,612,003A Plant derived cell culture material

1. A hydrogel comprising cellulose nanofibrils, wherein the cellulose nanofibrils are capable of being mechanically homogenized, chemically modified, or both, and wherein the cellulose nanofibrils are biocompatible, capable of being injected, and capable of forming a gel in situ.

2. The hydrogel of claim 1, wherein the chemical modifications are chosen from one or more of carboxymethylation, TEMPO oxidation, periodate oxidation, enzymatic treatment, or combinations thereof.

3. The hydrogel according to claim 1, wherein the cellulose nanofibrils are capable of being originated from a tunicate or tunicates, wood, a plant or plants, bacteria, algae, or combinations thereof.

4. The hydrogel according to claim 1, wherein the hydrogel is capable of being used for human or animal tissue repair, organ repair, tissue regeneration, organ regeneration, cell therapy, cancer treatment, or combinations thereof.

5. The hydrogel according to claim 1, further comprising one or more biopolymer, one or more synthetic polymer, or combinations thereof.

6. The hydrogel of claim 5, wherein the one or more biopolymer is chosen from one or more of alginate, hyaluronic acid, collagen, laminin, fibrin, dextran, gellan, and chitosan.

7. The hydrogel of claim 1, further comprising one or more of a pharmaceutical, a pharmaceutical compound, a pharmaceutical agent, a therapeutic compound, a therapeutic agent, or a drug.

8. The hydrogel of claim 7, wherein the hydrogel is injectable in a human or an animal to deliver the one or more of the pharmaceutical, the pharmaceutical compound, the pharmaceutical agent, the therapeutic compound, the therapeutic agent, or the drug, to a targeted area, body part, tissue, organ, or combinations thereof.

9. The hydrogel of claim 8, wherein the hydrogel is capable of a controlled release rate.

10. The hydrogel according to claim 1, further comprising one or more growth factor, one or more signaling molecule, or combinations thereof.

11. The hydrogel of claim 10, wherein the hydrogel is injectable in a human or an animal to deliver the one or more growth factor, the one or more signaling molecule, or the combinations thereof, to a targeted area, body part, tissue, organ, or combinations thereof.

12. The hydrogel of claim 11, wherein the hydrogel is capable of having a controlled release rate.

13. The hydrogel of claim 1, further comprising human or animal cells.

14. The hydrogel of claim 13, wherein the hydrogel is injected in a human or an animal to deliver the human or animal cells to a targeted area, body part, tissue, organ, or combinations thereof, and wherein the hydrogel is injected in a human or an animal to act as a scaffold.

15. The hydrogel of claim 1, wherein the hydrogel is bioconjugated with one or more adhesion protein, one or more other molecule affecting cell adhesion, or combinations thereof.

16. The hydrogel of claim 15, wherein the hydrogel is injected in a human or an animal in a targeted area, body part, tissue, organ, or combinations thereof, to attract cells, bind cells, act as a scaffold, or combinations thereof.

17. The hydrogel according to claim 1, wherein the hydrogel further comprises a dispersion comprising fibrils having a length between 0.05 and 20 μm.

18. The hydrogel according to claim 1, wherein the hydrogel comprises a dispersion having a solid content greater than 0.5% and less than 5% by weight.

19. A method of treating a human or an animal having a tissue or organ injury, wound, defect, pathology, or disease by injecting a hydrogel comprising cellulose nanofibrils, wherein the cellulose nanofibrils are capable of being mechanically homogenized, and wherein the cellulose nanofibrils are biocompatible, injectable, and capable of forming a gel in situ.

20. The method of claim 19, wherein the cellulose nanofibrils are capable of being originated from a tunicate or tunicates, wood, a plant or plants, bacteria, algae, or combinations thereof.
